(19) United States
(12) Patent Application Publication
(10) Pub. No.: US 2016/0339438 A1
(43) Pub. Date: Nov. 24, 2016

(54) True Nucleic Acid Amplification

(71) Applicant: Cyclone Biosciences LLC, Phoenix, AZ (US)

(72) Inventor: Stephen E. Griffin, Peoria, AZ (US)

(73) Assignee: Cyclone Biosciences LLC, Phoenix, AZ (US)

(21) Appl. No.: 15/226,296

(22) Filed: Aug. 2, 2016

Related U.S. Application Data
(63) Continuation of application No. 12/806,012, filed on Aug. 4, 2010.

(51) Int. Cl. B01L 7/00; C12Q 1/68; C12P 19/34

(52) U.S. Cl. CPC B01L 7/525; C12P 19/34; C12Q 1/686; B01L 2300/0864; B01L 2300/0867; B01L 2300/0874; B01L 2300/1872; B01L 2300/1883

(57) ABSTRACT

A system and method directed to DNA amplification with optional in situ purification, sequencing and/or detection, or a system compatible with integrated, post-amplification purification and or sequencing by capillary electrophoresis and other methods. The device is a single, helical channel formed of fused silica with heat zones defined about fixed arcs of the helix inner and/or outer circumference. The length of the helical channel and the cycle number and dwell time may be varied by altering the pitch of the helix within the cylindrical substrate. In another embodiment, the heat zone arcs lengths are also variable. In still another embodiment, multiple helical channels are available in parallel within the same structure. Separation channels may be integrated on the device for post-amplification purification and/or sequencing. One or more detection schemes may be provided on the device or seamlessly integrated with the device, for monitoring amplification and/or detecting specific products.

### CROSS-REFERENCE TO RELATED APPLICATIONS

This disclosure claims the benefit of priority to U.S. patent application Ser. No. 12/806,012, filed Aug. 4, 2010, the disclosure of which is incorporated herein in its entirety.

### BACKGROUND

The Polymerase Chain Reaction (PCR) technique (U.S. Pat. No. 4,683,202) and other related cyclic, polymerase-mediated reaction sequences have become a fundamental tool in biotechnology (e.g. forensics, medical diagnostics). PCR produces millions of copies of nucleic acid samples (DNA and RNA), typically beginning with a small number (even a single copy). PCR reproduction is typically achieved by 20 to 35 repeated cycles consisting of three steps: 1) template (sample) denaturation, 2) primer annealing to the template and 3) elongation mediated by a heat-stable DNA polymerase. While somewhat of a misnomer, this replication is generally termed “amplification” because each copy of nucleic acid (hereafter DNA), treated in small batches, doubles in number with each cycle: the DNA is reproduced geometrically. The conditions for PCR are well established in the art. While the parameters for DNA amplification by PCR and related methods are typically quite similar, some variations in the temperatures used, and the dwell times at those temperatures, is necessary to optimize the procedure for specific target samples and reagents.

In practice, commercial PCR techniques are batch processes. Samples are contained within small test tubes or microtiter plates (e.g. 96-well, 384-well) and are heated and cooled in situ. “Amplification” implies a continuous production stream, such as amplified sound produced by an audio amplifier not batch processes. While each batch of DNA in PCR is certainly reproduced on a massive scale, each cycle typically takes 1, 2 or more minutes for about a half an hour of total cycling time required to amplify the sample sufficiently. The number of cycles that a DNA sample may be subjected to, and therefore the maximum amplification achievable, is limited by the quantity of individual nucleotides available in the sample well or tube. Due to this limitation, if very large quantities of DNA are required, multiple batches are processed rather than simply extending the processing time by some number of cycles.

Early attempts to produce truly continuous PCR were based upon providing standard, linear capillaries (as know in the art and produced by Polymicro Technologies, for example) with sequential heat zones (constant temperature baths) along the length. The sample and reagents were passed through the capillary with the product collected out the opposite capillary terminus (Nakano et al., Biosci. Biotechnol. Biochem. 1994, 58, 349-352). A major problem with this approach was the length of the capillary that was required to provide the 60 to 100 individual heat zones required: small bore tubing of considerable length requires significant pressures to be applied in order to provide the necessary reagent flow.

A helical coil of capillary, wound about the three heat zones, would simplify continuous PCR in standard capillary, but the minimum coil diameters available using standard silica capillary remain larger than desirable, necessitating relatively long sections of capillary to achieve the desired number of cycles. The minimum coil diameter is limited by the high stresses imparted upon the capillary, in bending, and the relatively low long-term reliability of the materials in such tight coils. Attempts have been made to increase the tensile strength of capillary to permit tighter coiling (U.S. Pat. No. 6,902,759) or reduce the stresses imparted upon coiled capillaries (U.S. Pat. No. 5,552,042) but, to date, this work has failed to produce coils of diameters that are small enough to achieve the desired result of short path lengths for manageable applied pressures.

Although continuous DNA amplification is not required to reap myriad benefits from the technology, true amplification would have some definite advantages, e.g. in providing unlimited copies without parallel batch processing. Attempts have been made to more closely approximate true amplification by shortening cycle times and providing for more rapid changes in temperature, but with limited success and utility.

More recently, methods have been developed wherein the target DNA sample is passed through a channel, usually microfluidic (lab-on-a-chip in nature) with linear, serpentine or spiral channel architecture, wherein successive areas of the channel (s) are held at the three different temperatures needed for DNA amplification. As a result of the planar architecture of such devices, samples are necessarily subjected to nonfunctional temperature zones and total channel lengths remain high. Methods reported to date suffer reduced amplification efficiency, inflexible processing parameters, relatively high cost and significant back pressures (related to the total length of the microfluidic channel), sample dispersion, double helix formation post-denaturation, and cross-contamination between samples. Although some of these newer techniques are quite fast and are truly continuous, only linear channel architecture analogous to early capillary techniques are amenable to performing PCR in parallel.

Parallel PCR as exemplified by the work of Franzen, (U.S. Pat. No. 6,180,372), is desirable to minimize the velocity of flow within the capillary, thus reducing the eddy current mediated disruption of critical primer and base to template binding and dispersion, thereby improving amplification efficiency. Parallel PCR also promotes more efficient heat transfer through increased sample to heat source contact area, while delivering short total cycle times. The disadvantage of parallel methods is the increased interference and cross-contamination potential due to more sample-to-surface interaction as DNA tends to reversibly bind to most substrates used in microfluidic channel fabrication.

Capillary surface modification is used to address sample to channel adhesion problems, i.e. as known in the art of separation science (e.g. deactivation of glass surface with organosilanes). Cross-contamination issues in continuous PCR of multiple samples within a single channel have also been addressed by separating sample plugs within the capillary with oils (e.g. Nakayama et al., Anal. Bioanal. Chem., 2006, 386, 1327-1333), but the typically high viscosity of these oils exacerbates the back pressure problems of fluid flows inherent in small-bore channels.

Bidirectional flow microfluidic systems for PCR have also been proposed to minimize the problems associated with continuous flow devices (Chen et al., Anal. Chem, 2007, 79, 9185-9190). These devices show promise but are currently slower and less efficient than continuous and traditional methods, respectively, and offer less flexibility in application and varying thermal parameters.

Materials produced by batch and continuous PCR methods are typically impure, being at least contaminated with excess primer, nucleotides and enzyme: the product must usually be purified to be useful. It is also valuable to identify the product of PCR amplification (e.g. in medical genetics or diagnostic microbiology), although purification is not necessarily required if the product may be conclusively detected in the impure form (Chen et al., Lab Chip, 2007, 7, 14 13-1423).

It would be useful to provide a rapid, continuous or semi-continuous method for PCR with isolation from cross-contamination that is fast low cost, and permits parallel PCR without significant double helix formation while offering potential for integrating purification and/or identification of the product. It would be further useful if such a method were compatible with existing, highly parallel sample handling equipment, e.g. microtiter plates (MTPs) and MTP handlers.

### SUMMARY

Embodiments of the present invention are directed to DNA amplification with optional in situ purification and/or detection, or a system compatible with integrated, post-amplification purification and or sequencing by capillary electrophoresis and other methods. In the simplest embodiment, the device is a single, helical channel formed of fused silica with heat zones defined about fixed arcs of the helix inner and/or outer circumference. The length of the helical channel and, as such, the cycle number and dwell time, may be varied by altering the pitch of the helix within the cylindrical substrate. In another embodiment, the heat zone arcs lengths are also variable. In still another embodiment, multiple helical channels are available in parallel within the same structure. In further embodiments, separation channels are integrated on the device for post-amplification purification. In further embodiments, one or more detection schemes are provided for, on the device or seamlessly integrated with the device, for monitoring amplification and/or detecting specific products, e.g. specific DNA sequences.

The capillary described herein is intended either as a disposable cartridge or reusable device with a replaceable cartridge (depending upon the needs of the application) that is used within an instrument that provides for sample introduction, sample movement, thermostatically controlled heat zones of variable temperature and geometry, and separation and detection where desirable.

The cartridge is composed of a fused silica capillary, housed in a suitable housing, preferably polymeric or metallic, or more robustly the capillary is formed within a monolithic, cylindrical fused silica rod or the wall of a monolithic, cylindrical fused silica tube. The surrounding instrument may utilize technology that is well known in the art for fluid movement and temperature control, as well as separation and detection. Some embodiments of the instrument platform are unique, e.g., where heat is provided by infrared absorption of the reagents through the capillary wall, utilizing lasers or other infrared heat sources, rather than conductive heating.

This Summary is provided to introduce a selection of concepts in a simplified form that are further described below in the Detailed Description. This Summary is not indented to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

FIG. 1 depicts prior art as a serpentine channel, continuous flow PCR chip, as disclosed by Kopp, et al., Science, 1998, 280 1046-1048.

FIG. 2 depicts prior art as a spiral channel, continuous flow as disclosed by Jia, et al., Anal. Lett., 2005, 38, 2143-2149.

FIG. 3 depicts prior art as a parallel, linear channel, continuous flow PCR chip by Franzen, U.S. Pat. No. 6,180,372.

FIG. 4 depicts a single channel cartridge produced with commercially available, flexible fused silica capillary.

FIG. 5 depicts the cartridge in FIG. 4 installed within a heater block and with typical heat zones defined.

FIG. 6 depicts the simplest embodiment of the robust cartridge with a single channel formed in the wall of a fused silica tube.

FIG. 7 depicts the helical capillary monolith (HCM) in FIG. 6 installed within the heater blocks of a basic PCR instrument.

FIG. 8 depicts a four channel HCM variant for parallel PCR.

FIG. 9 depicts a single channel HCM with internal heating blocks.

FIG. 10 depicts the same HCM as in FIG. 9, heated externally.

FIG. 11 illustrates heating the HCM from the outside with radial optical fibers about the HCM circumference and length (latter not shown).

FIG. 12 illustrates the light source end of the fibers in FIG. 12 with a proposed illumination pattern.

FIG. 13 depicts a top view of a single channel HCM cartridge installed within a heater block.

FIG. 14 offers a side view of a single channel HCM cartridge with illumination/detection optical fibers and fluid connections.

Reference now will be made in detail to various aspects of this invention, including the presently preferred embodiments. Each example is provided by way of explanation of embodiments of the invention, not limitation of the invention. In fact, it will be apparent to those skilled in the art that various modifications and variations can be made in the present invention without departing from the spirit or scope of the invention. For instance, features illustrated or described as part of one embodiment can be used on another embodiment to yield a still further embodiment. Thus, it is intended that the present invention cover such modifications and variations within the scope of the appended claims and their equivalents.

### DETAILED DESCRIPTION OF ILLUSTRATIVE EMBODIMENTS

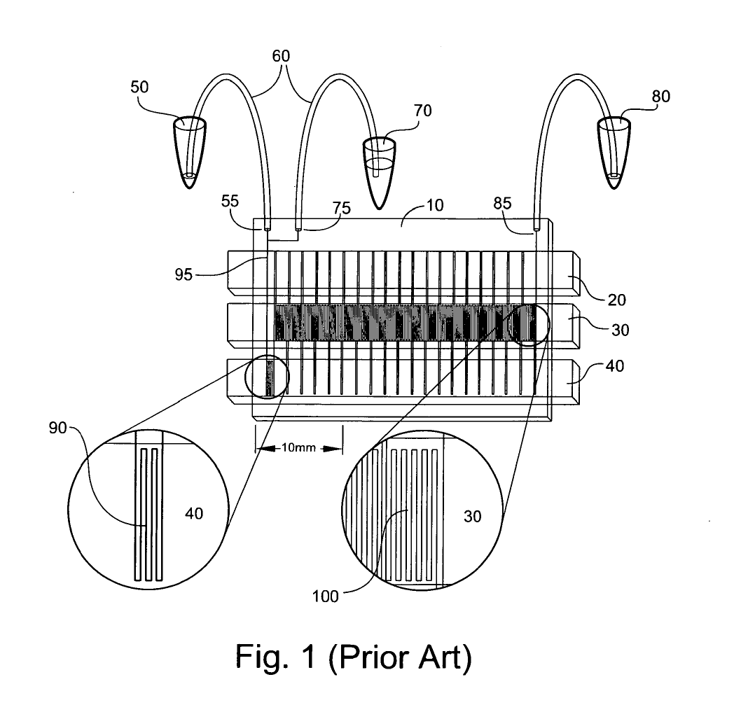

FIG. 1 illustrates prior art with a serpentine channel 95 formed in a glass plate 10 that is mounted on three heater blocks 20, 30, 40 held at the different temperatures required for denaturation 40, annealing 20 and elongation 30. The sample 50 is passed through a standard, capillary electrophoresis type, flexible fused silica capillary (available from Polymicro Technologies, Phoenix, Arizona) 60 onto the glass lab chip inlet at 55. Buffer 79 is passed through a similar capillary 60 to inlet 75 of the chip. The sample is passed through the serpentine channel 95, first at annealing temperature 20 and elongation temperature 30 (both unnecessary for the amplification cycle) before it reaches the denaturation heater block 40 where, in the first cycle (see detail circle) it dwells three times as long 90 as in the subsequent cycles (see Koop for reasoning). The sample then passes, again unnecessarily, through elongation temperature 30 to reach annealing temperature 20, and then reverses to pass back to the elongation block 30 where it dwells for considerable time due to the multiple loops at 100 in the detail circle. Shorter denaturation zone cycles then repeat 19 more times until the sample exits the chip at the outlet port 85 for collection in the product reservoir 80.

It is readily apparent that many compromises must be made in such an arrangement. Much of the sample is treated at temperatures not in keeping with standard PCR protocols since the capillary has to pass over undesired heat zones to reach those that are desired. Further, while the dwell times at the individual temperature blocks can be controlled by adding or subtracting channel loops, this can only be accomplished at considerable expense since the glass chip is formed by photolithography methods that are costly and time consuming. The chip itself is costly to produce and has a relatively large footprint for a microchip, even while providing a minimum number of cycle repetitions.

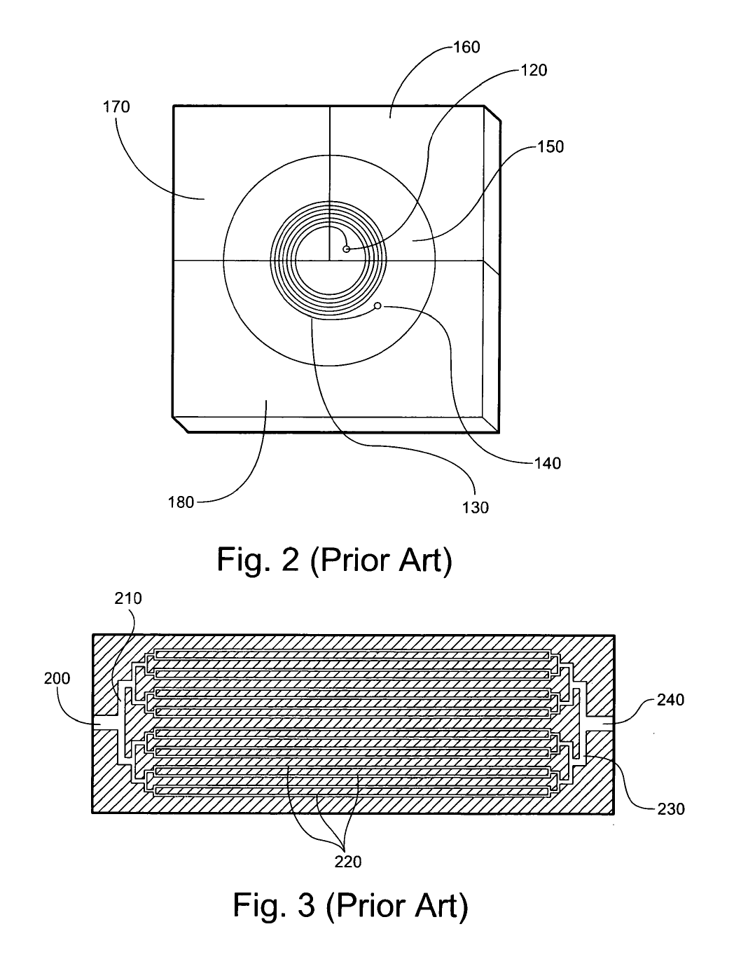

FIG. 2 illustrates an alternative microchip format where the channel 130 is a spiral. The sample and product “real world” interface is similarly accomplished through an inlet 120 in the glass or polymer chip 150 and outlet 140. The sample passes through the heat zones for denaturation 160, annealing 170 and elongation 180 in the proper order in this arrangement, but altering dwell times within zones is only available through altering flow rates or the heater block geometries, and the thermal dwell times differ for each cycle (as the channel circumference increases within the spiral).

FIG. 3 illustrates a linear chip format with 16 parallel channels 220. Heater blocks (not shown) are positioned below an above the polymer microchip. The sample is introduced at 200 where it is distributed by a manifold 210 into the parallel channels 220. The rate of flow within the device is much slower than for single channel devices, reducing diffusion and improving annealing efficiency. Product is collected in a second manifold 230 and delivered through the outlet port 240.

Again, the shortcomings of the device are related to the costs of changing the chip geometry and heater geometry and the device continues to have a large footprint for a microchip device. Further, the heater block geometry is necessarily quite complex with three zones needed for each cycle of denaturation, elongation and annealing desired.

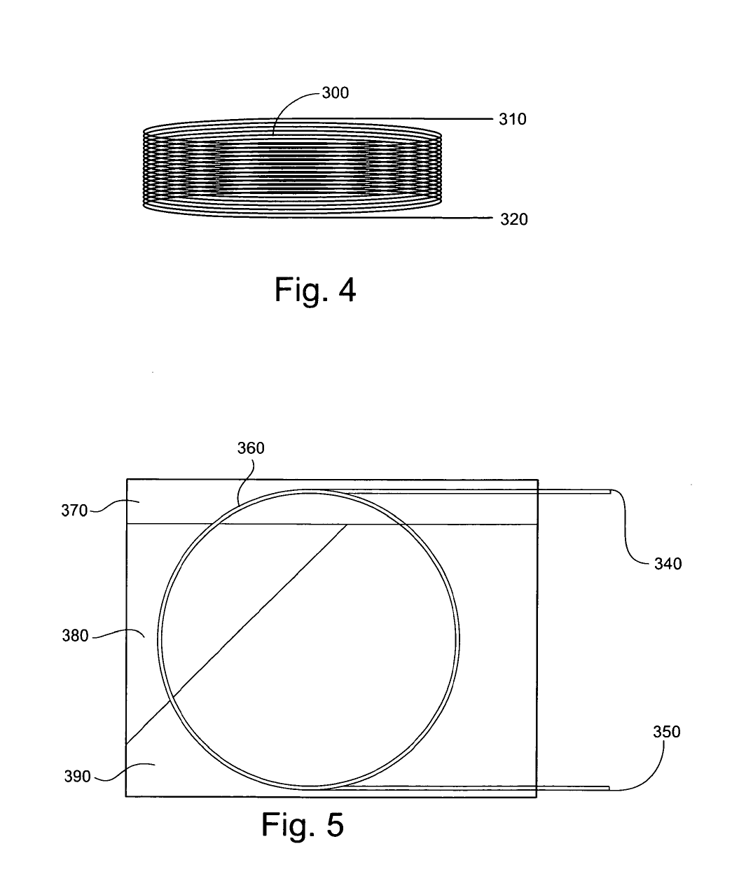

FIG. 4 is a simple illustration of a small capillary coil, constructed of the same material used for fluidic coupling in prior art (e.g. 60 in FIG. 1): standard CE-type capillary. While such a coil 300 may function for the invention described herein, it is less than optimum in that the minimum coil diameter is very limited (˜2 cm) due to tensile strength limits of CE capillary. The coil serves, however, as an easily envisioned illustration of the concepts discussed herein, and should capillary manufacturing technology advance to a point where long term reliability is achievable in extremely tight coils, such a system could be functional.

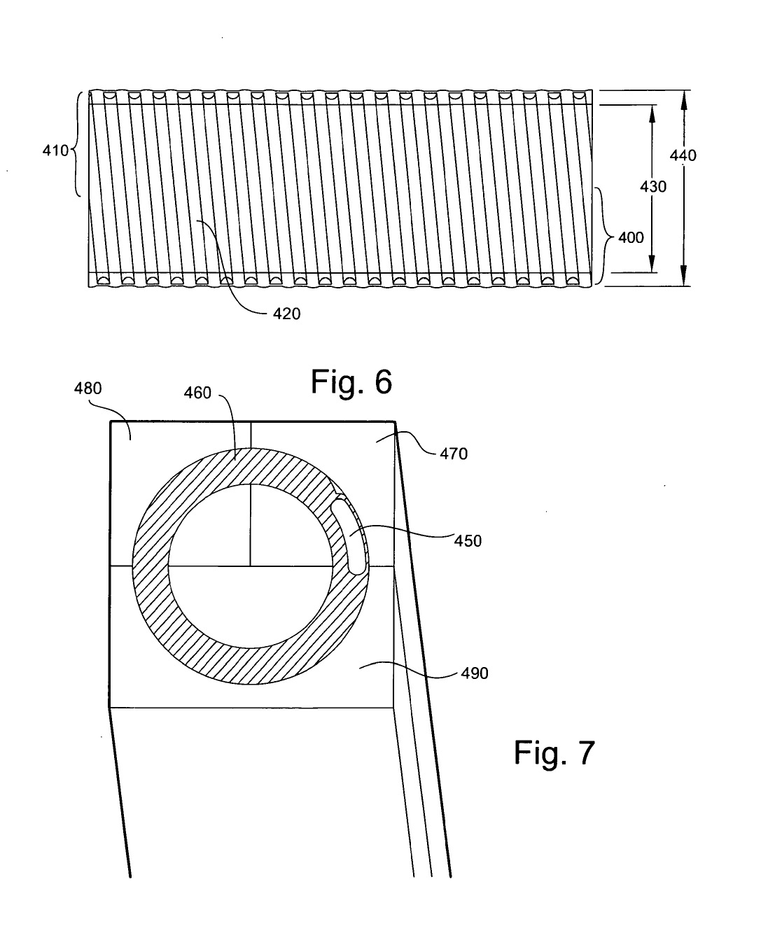

Such a coil could be mounted in an annular space machined within similar heater blocks as those used in FIG. 2, as illustrated in FIG. 5. Sample is introduced (more easily than in prior art directly into the capillary at 340 to access the coil 360. It passes through a denaturation zone block 370, to the annealing zone block 380 and on to the elongation zone block 390 and then repeats this temperature cycle. Each cycle is the same since the coil is cylindrical rather than spiraled, as in the prior art illustrated in FIG. 3. Product is collected directly from 350.

While solving some problems with prior art, e.g. permitting parallel processing, maintaining equivalent cycles, simplify fluidic interface, the device depicted in FIG. 5 remains relatively inflexible to modification of dwell times in individual temperature zones, although such coils would be far less costly to produce than polymer and glass microchips.

FIG. 6 is a sketch of the simplest embodiment of the capillary to be applied to the preferred art disclosed herein: what the inventor calls a Helical Channel Monolithic (HCM) column. In essence it is merely a small glass tube (preferably synthetic fused silica of low inherent chemical activity) with an internal diameter 430, on the order of 1.5 mm to 9.5 mm, and an outer diameter 440, on the order of 2 mm to 10 mm although these dimensions are by no means limitations of the technology. A capillary channel 420 is formed within the wall of the tube by low cost processes (as disclosed in U.S. Pat. No. 7,469,557); potentially lower cost than that for producing standard CE capillary (for equivalent channel bore and length).

It is possible, and preferable to produce tighter coils 420 than illustrated in FIG. 6, but looser coils are simpler to illustrate with clarity. It is also possible to produce inlets 400 and outlets 410 that are orthogonal cross-sections of the channel axis by eliminating the pitch altogether at the device ends, but the much larger openings provided by cutting across the coil pitch may be useful in some applications for reducing electrical field gradients, e.g. in electroosmotic flow (EOF) driven devices. It is also possible to produce the helical channel within a monolithic glass rod, rather than the wall of a tube, and to provide the inlet and outlets at the center of the ends of the rod, which may be useful for simplified coupling of modular sections of HCM material.

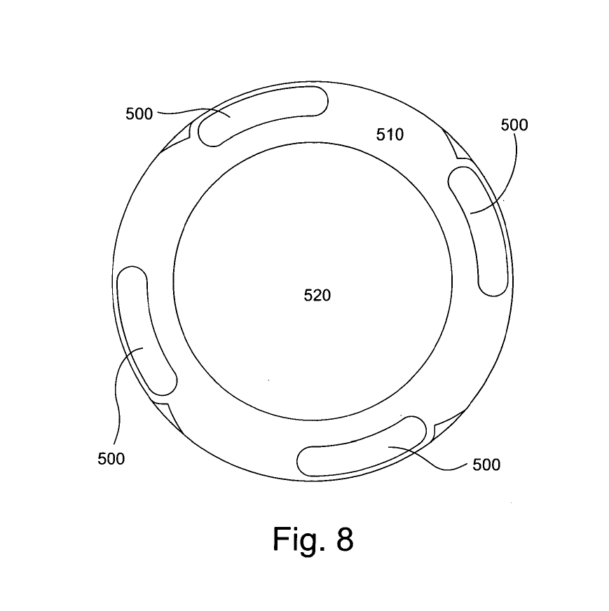

FIG. 7 depicts the single channel HCM 460 of FIG. 6 mounted within a three heat zone 470, 480, 490 block within which an annular space has been machined to accommodate the HCM. Only the inlet 450 appears in the figure, as the outlet is at the opposite end of the rectangular, multizone heater block. Parallel processing can easily be accommodated by replacing a single channel HCM with a multi-channel HCM, as depicted in FIG. 8 where the wall of the silica tube 510 has four channels 500 machined in parallel. Multilayered devices are also possible to construct, as disclosed in U.S. Pat. No. 7,469,557, permitting return flow in the opposite direction of the initial flow to permit all fluidic connections to be accomplished in a single plane or block. Further in multichannel HCM, different channels may have different internal diameters.

While the art disclosed herein represents a useful advance in PCR speed and miniaturization, it is apparent that the inflexibility of individual dwell times in zones remains for individual HCM cartridges mounted in fixed-zone geometry heater blocks as depicted in FIG. 7. It is important to point out, however, that HCM cartridges with different pitches, channel counts and lengths are easily and cheaply constructed, greatly reducing this problem. Coupled with the expanded range of flow rates permitted by the relatively short capillary lengths afforded by the HCM geometry, sufficient flexibility in sample dwell times may be possible.

Even so, it would be desirable to provide a means of altering dwell times in zones without changing HCM geometries or altering the heat zone blocks. Further, for very small HCMs, it would be desirable to provide an mean of reproducibly heating the bore of the device (e.g. 520 in FIG. 8) in the very small dimensions afforded.

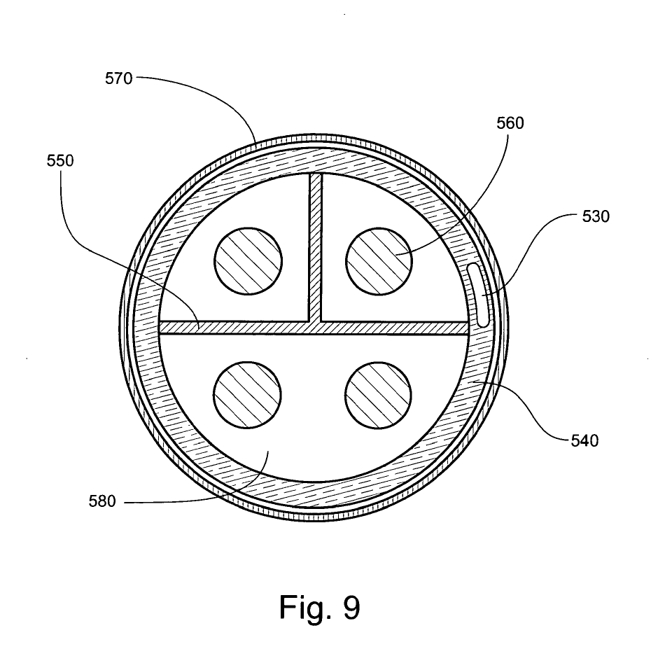

Fiber optics offer the potential to deliver energy into small, confined spaces, such as that present in the bore of small HCMs. FIG. 9 depicts an HCM cylinder 540 with a single channel (inlet 530). Within the bore of the overall HCM monolith is disposed a reflective barrier or insulator 550 that defines the three thermal zones typical of PCR. Within the zones are disposed at least one, preferably four, diffusing optical fibers 560 which deliver different amounts of energy to each zone. Surrounding the HCM is a cylindrical reflector 570. The diffusing optical fibers might also be replaced by very small cartridge heaters or other heating elements. The zones 580 between the optical fibers 560 may also be filled with static or flowing fluid for enhanced heat transfer and zone uniformity or, alternatively, the spaces about the diffusing fibers could be empty such that the sample is heated by intrinsic absorption of the radiant light energy by the sample within the helical channel(s).

The optical fibers can be illuminated with a common light source, through use of attenuators to control the light emitted by each fiber or by separate sources and even differing wavelengths.

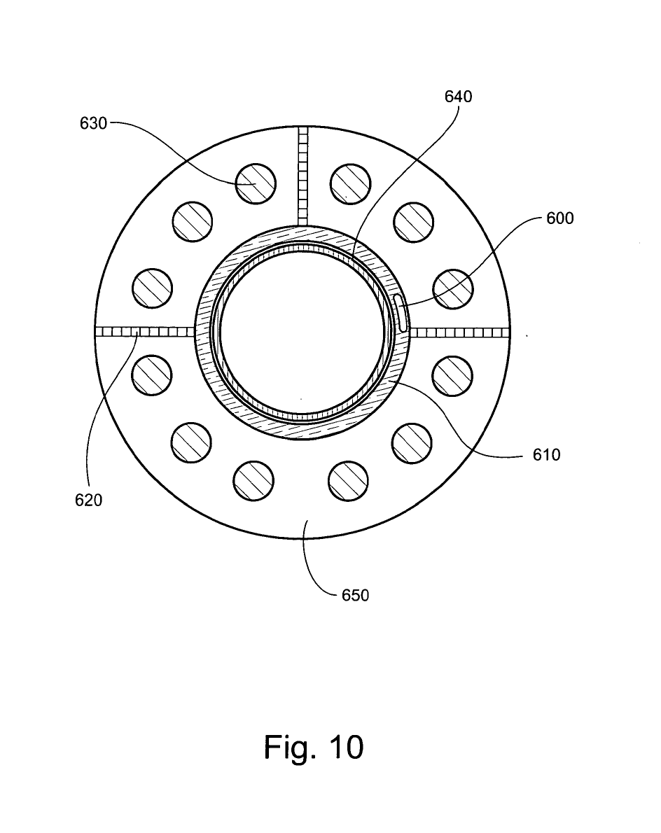

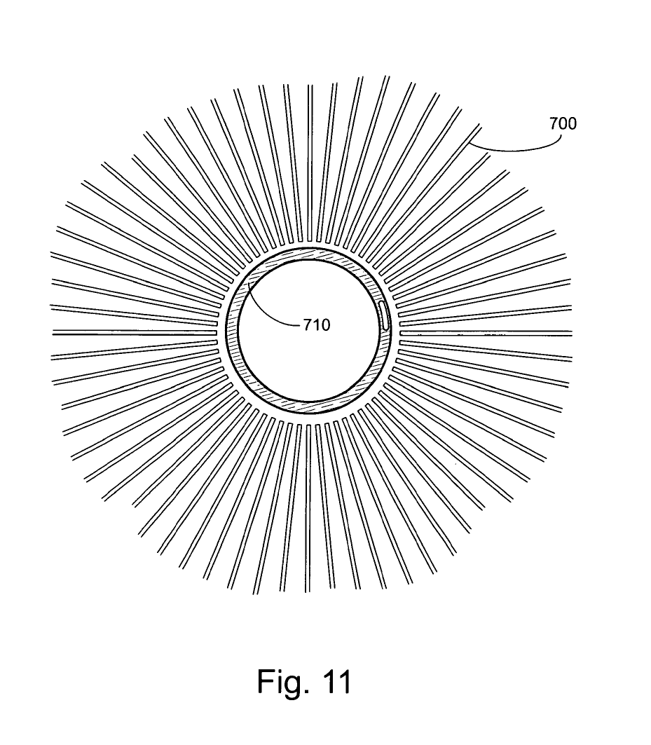

FIG. 10 depicts a similar arrangement as that found in FIG. 9, but with the diffusing optical fibers 630 disposed in zones about the outer diameter of the HCM 610. In this embodiment, a reflective barrier or insulator 620 lies outside the HCM and the cylindrical reflector 640 is disposed within the HCM bore. Again, fluid may be added to the spaces 650 about the fibers 630 in the individual zones to aid in heat transfer and heat uniformity throughout the zone, and the fibers 630 could be replaced with cartridge heaters or other heat sources known to those familiar with the art.

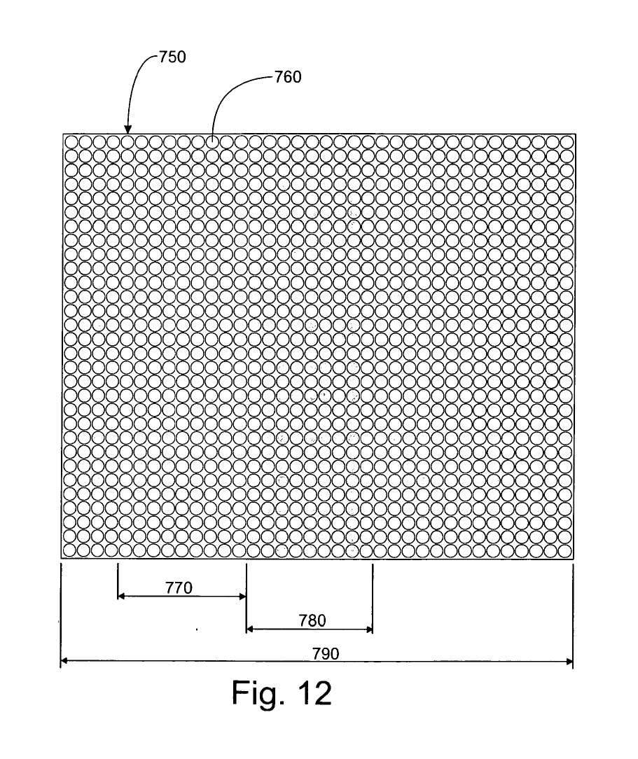

FIG. 11 depicts an alternative embodiment for providing heat zones about the circumference of the HCM 710, where a number of optical fibers are disposed about the outer diameter and length of the HCM 710, each potentially delivering a defined amount of energy to the small portion of the monolith that it illuminates. FIG. 12 shows these fibers 760 terminated at the opposite end, the illumination or light source end, where the circumferential fibers inputs are arranged in a plane with a width 790 equivalent to the circumference of the HCM. 750 marks the point where the inlet to the HCM is located with flow proceeding to the right and down in the drawing. The first nine fibers along the channel path through the HCM are colored to represent denaturation temperature so this is the initial denaturation zone 770. The next nine fibers in the line define the annealing zone 780 for the first coil of the HCM and are colored to indicate that zone. The remaining fibers in the first row and the first four of the second row define the elongation heat zone.

By altering the number of fibers supplied with a particular energy, one may alter the dwell time for samples within the zones by altering the length of the zone. For example, were the whole first row of fibers maintained at denaturation energy, the extended dwell at denaturation in the first cycle, illustrated in the prior art depicted in FIG. 1 could be approximated. This method of providing energy to the HCM therefore permits a great deal of freedom in selection of zone temperatures and dwell times, unavailable in prior art. If inherently stable sources are used to illuminate the individual fibers supplying energy to the HCM, e.g. feedback stabilized lasers or infrared diode lasers, the temperature control is very accurate and precise. An additional advantage of this heating method may be a significant reduction of the mass of material that must be maintained at constant temperature, through the reduction or elimination of heat absorbed by the monolithic column itself.

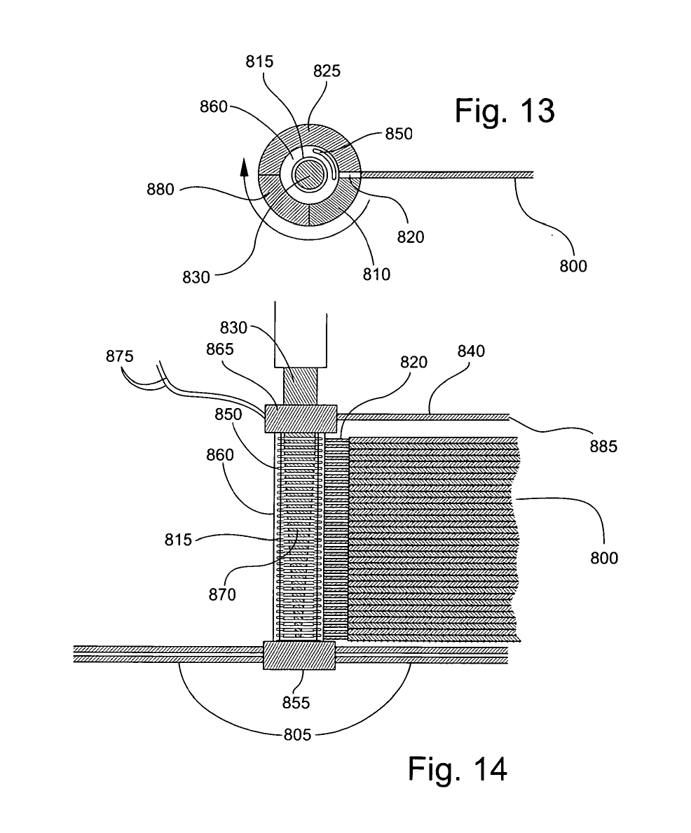

FIGS. 13 and 14 illustrate additional features enabled by the simple geometry of the HCM, with some components removed from the TOP (FIG. 14) and SIDE (FIG. 15) views for clarity. The TOP view depicts the HCM 860, with the helical channel (outlet) 850, mounted within a divided, cylindrical heater block offering three temperatures corresponding to denaturation 880, elongation 825 and annealing 810. The arrow depicts the direction of fluid flow within the device, rising up the helical channel from below.

In this embodiment (FIGS. 13 and 14), a tapered 870 diffusing optical fiber 830, disposed inside the bore wall 815 of the HCM monolith, provides light energy to the flowing sample throughout the device, for illumination or excitation of fluorescence. Fluorescence or other optical signals are detected at each turn 850 of the helical channel by a linear array of receiving optical fibers 800.

Fluidic input of the sample, buffer stream and reagents are provided to a lower or inlet manifold 855, depicted in the SIDE view, via capillaries 805, and PCR product is collected in an upper or outlet manifold 865 for recovery via capillary 885. By providing electrical connection 875 to the PCR product within manifold 865, and at the outlet of the recovery capillary 885, electrophoretic-type separation of the PCR products may be accomplished without disruption of the continuous amplification provided by the core device. Alternatively, recovery capillary 885 may connect to, or itself embody, a second device designed for separation of PCR products from the sample solution by other means, such as solid phase extraction or monoclonal antibody affinity.

The preferred embodiment of the invention provides direct heating of the sample within the HCM via optical absorption or light energy and continuous monitoring of PCR progress via fluorescence detection of products at each coil or each completed cycle. By extending the length of the HCM, either by adding additional turns of the helical channel or by means of alternative geometries, including but not limited to microfluidic circuits as known in the art but disposed within the cylindrical geometry described herein, and by providing electrical connections to the fluidic channels at manifolds or accessory ports, electrophoretic-type separations of PCR products may be performed in situ, without any additional handling of the PCR products. One may also explore providing electrical connections across the amplification helix itself, for control of the distribution of sample components within the helical channel during amplification or during pauses in flow or altered flow rate or direction within the channel: parameters that are completely unavailable in the batch PCR processes in commercial use.

The compact and cylindrical geometry of the HCM-based devices enables those familiar with the art to envision massively parallel applications utilizing arrays of HCMs arranged in a grid to mate with MTPs (absent fluidic inlet manifolds). Where the small diameters of the HCM cartridges are essentially preserved by utilizing compact methods of providing heat zones, e.g. HCM bore-based fiber optic heating, compatibility for simultaneous address of each well in standard 384-well, or possibly 1536 well MTPs may be achieved.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications and/or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A method of conducting a Polymerase Chain Reaction (PCR) amplification, the method comprising:
providing a Nucleic Acid Amplification Device (NAAD), the NAAD comprising a cartridge that includes a helical channel monolith which is a fused silica rod or tube having a helical capillary channel extending within the fused silica rod or tube from an inlet end to an outlet end; and
three heaters, each adjacent to the fused silica rod or tube, extending a length of the fused silica rod or tube from the inlet end to the outlet end, and extending in arcs about the fused silica rod or tube, wherein the three heaters divide the fused silica rod or tube into three different thermal zones that, individually, extend from the inlet end to the outlet end of the fused silica rod or tube;
differentially heating the three thermal zones of the NAAD thereby providing a denaturation zone, an annealing zone, and an elongation zone;
introducing a mixture of a nucleic acid sample and PCR reagents to the helical capillary channel; then
repeatedly cycling the mixture of the nucleic acid sample and PCR reagents through the denaturation zone, the annealing zone, and the elongation zone, wherein each cycle has the same dwell time; and then
collecting a product of the PCR amplification.

2. The method of claim 1 further comprising monitoring each cycle of the PCR amplification.

3. The method of claim 2, wherein the cartridge includes a fused silica tube; and the NAAD further includes a diffusing optical fiber disposed inside the fused silica tube; a longitudinal detecting zone positioned between two thermal zones; and a linear array of receiving optical fibers disposed outside the helical channel monolith and longitudinally along the detecting zone, each optical fiber adjacent to a turn on the helical capillary channel, and adapted to collect light energy from the detecting zone;
the method further comprising:
collecting light energy from the detecting zone; and
detecting fluorescence or another optical signal at each cycle of the PCR amplification.

4. The method of claim 3, wherein the diffusing optical fiber is tapered.

5. The method of claim 3 further comprising exciting fluorescence of a sample within the helical capillary channel; and
detecting the fluorescence via the receiving optical fibers.

6. The method of claim 1 further comprising purifying the product of the PCR amplification.

7. The method of claim 6, wherein the PCR amplification product is purified via capillary electrophoresis.

8. The method of claim 6, wherein the PCR amplification product is purified via transport within the helical capillary channel.

9. The method of claim 1 further comprising replacing the cartridge after collecting the product of the PCR amplification.

10. The method of claim 1 further comprising providing a new cartridge prior to differentially heating the three thermal zones of the NAAD.

11. The method of claim 1 further comprising disposing of the cartridge after collecting the PCR amplification product.

12. The method of claim 1, wherein the helical capillary channel is carried within a wall of the rod or tube.

13. The method of claim 1, wherein the heaters, individually, include a diffusing optical fiber capable of delivering energy to a thermal zone; and the NAAD further comprising: a cylindrical reflector disposed about the cartridge.

14. The method of claim 13, wherein the helical channel monolith includes a fused silica tube; wherein the cylindrical reflector is disposed within the fused silica tube; and wherein the diffusing optical fibers are disposed outside the fused silica tube.

15. The method of claim 13, wherein the helical channel monolith includes a fused silica tube; wherein the diffusing optical fibers are disposed within the fused silica tube; and wherein a reflective barrier or insulator divides the heaters.

16. The method of claim 1, wherein the heaters are block heaters; and wherein the block heaters had been machined to provide an annular space which accommodates the cartridge.

17. The method of claim 1 further comprising a multizone heater block that includes the three heaters; wherein the cartridge is mounted within an annular space machined in the multizone heater block.
